(19) United States
(12) Patent Application Publication
(10) Pub. No.: US 2006/0129152 A1
(43) Pub. Date: Jun. 15, 2006

(54) Absorbable Anchor for Hernia Mesh Fixation

(76) Inventor: John Isbell Shipp, Jacksonville Beach, FL (US)

Correspondence Address: JOHN I. SHIPP, PO Box 3614, PONTE VEDRA BEACH, FL, 32004, US

(21) Appl. No.: 10/905,020

(22) Filed: Dec. 10, 2004

(51) Int. Cl. A61B 17/58

(52) U.S. Cl. 606/72

(57) ABSTRACT

A method of forming and deploying an absorbable anchor for hernia mesh fixation is disclosed. The diameter of the anchor is reduced upon insertion to minimize entry hole size and insertion force and is increased when urged proximal. The anchor is formed from co-polymers of lactide and glycolide.

The present application claims priority to U.S. patent application Ser. No. 10/709,297, the entire contents of which are hereby incorporated by reference.

### BACKGROUND OF THE INVENTION

This invention relates to surgical fasteners and their associated applicators, and more particularly, surgically fastening material to tissue and their method of use.

In laparoscopic repair of hernias surgical fasteners have been used to attach repair mesh over the hernia defect so that bowel and other abdominal tissue are blocked from forming an external bulge that is typical of abdominal hernias. The role of the fasteners is to keep the mesh in proper position until tissue ingrowth is adequate to hold the mesh in place under various internal and external conditions. Adequate ingrowth usually takes place in 6-8 weeks. After that time the fasteners play no therapeutic role. Fixation anchors comprise a mesh fixation feature, or head, a mesh-tissue interface section, and a tissue-snaring feature that holds the anchor in place under force developed within the body.

At present, there are a variety of surgical devices and fasteners available for the surgeon to use in endoscopic and open procedures to attach the mesh patch to the inguinal floor. One such mesh attachment instrument uses a helical wire fastener formed in the shape of a helical compression spring. Multiple helical wire fasteners are stored serially within the 5 mm shaft, and are screwed or rotated into the mesh and the overlaid tissue to form the anchor for the prosthesis. A load spring is used to bias or feed the plurality of helical fasteners distally within the shaft. A protrusion extends into the shaft, while preventing the ejection of the stack of fasteners by the load spring, allows passage of the rotating fastener. U.S. Pat. Nos. 5,582,616 and 5,810,882 by Lee Bolduc, and 5,830,221 by Jeffrey Stein describe instruments and fasteners of this type.

U.S. Pat. Nos. 5,203,864 and 5,290,297 by Phillips describe two embodiments of a hernia fastener and delivery devices. One of the Phillips fasteners is formed in the shape of a unidirectional dart with flexible anchor members. The dart is forced through the mesh and into tissue by a drive rod urged distally by the surgeon's thumb. The anchor members are forced inward until the distal end of the dart penetrates the overlaid tissue and then the anchor members, presumably, expand outward without any proximal force on the dart thus forming an anchor arrangement. This requires an extremely forceful spring force generated by the anchor members. Multiple darts are stored in a rotating cylinder, much like a revolver handgun.

Phillips second fastener embodiment is a flexible H shaped device. The tissue penetrating means is a hollow needle containing one of the legs of the H. The H shape is flattened with the cross member and the other leg remaining outside the hollow needle owing to a longitudinal slot therein. A drive rod urged distally by the surgeon's thumb again delivers the fastener. The contained leg of the H penetrates the mesh and tissue. After ejection the fastener presumably returns to the equilibrium H shape with one leg below the tissue and one leg in contact with the mesh with the cross member penetrating the mesh and the tissue, similar to some plastic clothing tag attachments. Phillips depicts the installed device returning to the H shape but he fails to teach how to generate enough spring action from the device to overcome the high radial forces generated by the tissue.

A series of U.S. Pat. Nos. 6,572,626, 6,551,333, 6,447,524, and 6,425,900 and patent applications 200200877170 and 20020068947 by Kuhns and Kodel, all assigned to Ethicon, describe a super elastic, or shape metal fastener and a delivery mechanism for them. The fasteners are stored in the delivery device in a smaller state and upon insertion into the mesh and tissue transitions to a larger anchor shaped state. The Ethicon fastener is delivered by an elaborate multistage mechanism through a hollow needle that has penetrated the mesh and the tissue. The hollow needle is then retracted to leave the fastener to change shape to a more suitable configuration for holding the mesh in place.

The primary problem with these prior art fasteners is that the mesh is attached to body tissue in as many as 100 places for large ventral hernias. This results in a large quantity of metal remaining in the body as permanent implants, even though after the ingrowth phase the fasteners serve no useful purpose. Compounding this problem the distal ends of the fasteners are sharp pointed and thus pose a continued pain or nerve damage hazard.

One alternative to metallic fixation devices is bio-absorbable materials. These materials are degraded in the body by hydrolysis. After degradation the body metabolizes them as carbon dioxide and water. These materials require special attention to many design details that are much more demanding than their counterparts in metallic fixation devices such as applicator tool design, sterilization processes, and packaging. Metallic tacks or anchors provide structural strength that simplifies their insertion and since the materials, usually titanium or nickel-titanium alloys (shape metal), are chemical and radiation resistant and are very temperature tolerant many options are available to the designer. Not so for bio-absorbable materials.

The basic considerations of an effective mesh fixation applicator and anchor are the material strength, absorption time, the sterilization method, and packaging requirements. The ease of insertion of the anchor through the mesh and into the tissue, the ease of ejecting the anchor from the tool, the fixation strength of the anchor once implanted, the time required after insertion for the anchor to be degraded and metabolized by the body are all effected by the choice of anchor material, the geometry of the design, and the forming process.

Materials of appropriate strength are generally limited to synthetic materials. Currently, the FDA has cleared devices made from polyglycolide (PG), polylactide (PL), poly caprolactone, poly dioxanone, trimethylene carbonate, and some of their co-polymers for implant in the human body. These materials and their co-polymers exhibit a wide variation of properties. Flex modulus ranges from a few thousand to a few million PSI, tensile strength ranges from 1000 to 20,000 PSI, in vivo absorption times range from a few days to more than two years, glass transition temperatures range from 30-65 degrees centigrade, all with acceptable bio-responses. Unfortunately, however, the optimum values of each of these properties are not available in any one of these materials so that it is necessary to make performance tradeoffs.

Mechanical Properties

Most hernia mesh fixation devices are used in laparoscopic hernia repair. In general laparoscopic entry ports have been standardized to either 5 or 10 mm (nominal) diameter. In the case of prior art of metallic, fixation devices, 5 mm applicators are universally employed. Since it is not clear that the medical advantages of the use of absorbable anchors would totally out weigh the disadvantages of moving to a 10 mm applicator it must be assumed that absorbable anchors must also employ 5 mm applicators. Because of the lower strength of absorbable material this requirement imposes severe design constraints on both the applier and the anchor.

After successful insertion there are two ways for a fixation anchor to fail. It can fracture, separating the mesh holding feature from the tissue-snaring feature, or it can pull out of the tissue owing to inadequate tissue snaring. Increased forces are placed on the anchor during sudden elevations of intra-abdominal pressure (IAP) caused by straining, coughing or the Valsalva maneuver, a medical procedure whereby patients close their nose and mouth and forcibly exhale to test for certain heart conditions. The later can generate an IAP of up to 6.5 PSI. For nonporous mesh and a hernia area of 50 square centimeters, for example this increased IAP places 50.3 pounds of force on the anchors fixating the mesh. Typically 40 anchors would be used to secure the hernia mesh of 150 square centimeters so that each anchor would, at this elevated IAP, experience approximately a 1.26-pound tensile force on the mesh-retaining feature and the tissue-snare feature. The tensile strength between these two features and the tissue snare force must exceed this force generated by the increased IAP or else the mesh fixation can fail.

The strength and flexibility of the anchor material are of major importance in the design considerations of the applicator, particularly in the case of anchors formed from polymers. Ory, et al (U.S. Pat. No. 6,692,506) teaches the use of L Lactic Acid polymer. Ory discloses adequate fixation strengths but the applicator device required to insert his anchor is necessarily 10 mm in diameter thereby causing the procedure to be more invasive than necessary. Ory further discloses a hollow needle with a large outside diameter, through which the anchor is inserted, that forms a rather large hole in the mesh and tissue to supply adequate columnar strength for penetration of the anchor. Entry holes of this size can give rise to multiple small hernias know as Swiss cheese hernias.

Absorption Time

There are two forms of PL, one synthesized from the d optical isomer and the other from the I optical isomer. These are sometimes designated DPL and LPL. A polymer with 50-50 random mixture of L and D is herein designated DLPL.

High molecular weight homo and co-polymers of PG and PL exhibit absorption times ranging from 1 month to greater than 24 months. Homo crystalline PG and PL generally require greater than 6 months to absorb and thus are not optimum materials for hernia mesh fixation. Amorphous co-polymers of PG and PL, on the other hand, typically degrade in less than 6 months and are preferably used in the present invention. For high molecular weight co-polymers of PG and PL the actual absorption time is dependent on the molar ratio and the residual monomer content. For a given monomer residual the absorption time varies from about 1 month to about 5 months as the molar content of DLPL increases from 50 to 85 percent with PG decreasing from 50 to 1 5 percent. Co-polymers of DLPL and PG in the molar range of 50 to 85 percent of DLPL are preferred for this invention. The geometry of the anchor also effects the absorption time. Smaller high surface area devices absorb faster.

The time required for the body to react to the foreign body of the mesh for tissue ingrowth into the mesh is typically 10 days. However, mesh migration and mesh contraction can occur for more than two months if not adequately stabilized. Since fixation anchors can impinge upon nerves and cause pain it is desirable for the anchors to be absorbed as soon as possible after the tissue ingrowth and after the mesh is secure against migration or contraction. For most absorbable materials there is a difference between the time for loss of fixation strength and mass loss. Fixation strength decreases quicker than anchor mass owing to some degree of crystalline structure in the polymer. For these reasons the preferred absorption time for the current invention is 3-5 months after implant.

Temperature Effects

Glass transition temperature (Tg) is the temperature above which a polymer becomes soft, can loose its shape, and upon re-cooling can shrink considerably. Both crystalline and amorphous polymers exhibit glass transitions in a temperature range that depends on the mobility of the molecules, which is effected by a number of factors such as molecular weight and the amount of residual monomers. Glass transition temperatures range from about 43 to 55 degrees centigrade (deg. C.) for co-polymers of PG and DLPL. Where as 100% PG has a Tg of 35-40 deg. C. and 100% PL exhibits a Tg from 50-60 deg. C. Since the core temperature of the body can reach 40 degrees C. the preferred Tg for the material comprising the current invention is greater than 40 deg. C. In addition hernia mesh anchors are often manufactured and shipped via surface transportation under uncontrolled, extreme heat conditions. Temperatures in commercial shipping compartments in the summer can exceed 60 degrees C. It is necessary then to provide thermal protection in the packaging so that the anchor temperature does not exceed its Tg.

Sterilization and Packaging

Bio-absorbable polymers degrade when exposed to high humidity and temperature. Autoclaving cannot be used, for example. Most ethylene oxide (ETO) sterilization processes employ steam and high temperatures (above Tg) to obtain reasonable “kill” times for the bio-burden commonly found on the device. High doses of gamma radiation or electron beam radiation (E Bream), both accepted methods of sterilization for many devices, could weaken the mechanical properties of PG, PL and their co-polymers. It is therefore necessary during the manufacturing process of the anchor and its applicator to maintain cleanliness to a high degree such that the bio-burden of the components is small enough so that pathogens are adequately eradicated with less severe forms of sterilization.

Radiation doses above 25 kilogray (kgy) are known to lessen the mechanical strength of bio-absorbable polymers whereas some pathogens are known to resist radiation doses below 10 kgy. It is therefore necessary, for the preferred embodiment of the present invention during manufacturing to keep the pathogen count below a certain threshold to insure the accepted regulatory standards are met for radiation levels between 10 and 25 kgy.

In a second embodiment of the present invention it is necessary during manufacturing to keep the pathogen count below a certain threshold to insure the accepted regulatory standards are obtained for sterilization using a non-steam, low temperature, ethylene oxide (ETO) process below Tg of the anchor polymer.

Anchors of the present invention must be carefully packaged to maintain adequate shelf life prior to use. Care must be taken to hermetically seal the device and to either vacuum pack, flood the package with a non-reactive dry gas prior to sealing, or to pack the device with a desiccant to absorb any water vapor since hydrolysis breaks down the backbone of the co-polymers.

ETO sterilization requires the gas to contact the device to be sterilized. Devices that are not humidity sensitive can be packaged in a breathable packaging material so that ETO can diffuse in, and after sterilization, diffuse out so that the device can be sterilized without unsealing the packaging. For the alternate embodiment of the present invention the device must be hermetically sealed after sterilization with ETO. Since gamma radiation and electron beam radiation sterilization can be accomplished through hermetically sealed packaging without disturbing the seal, either of these two sterilization processes is employed for the preferred embodiment of the present invention.

Ory, et al (U.S. Pat. No. 6,692,506), Criscuolo, et al (US application 20040092937), Phillips (U.S. Pat. Nos. 5,203,864 and 5,290,297), Kayan (U.S. application 20040204723), and Shipp (U.S. application Ser. No. 10/709,297) have suggested the use of bio-absorbable materials for use as hernia mesh fixation devices to solve the problems associated with the permanency of metal implants. Ory, preferably, suggests forming the fixation device from LPL but the absorption time for LPL can exceed two years, much longer than optimum for hernia fixation devices since the lessening of pain depends on mass loss of the device. While Phillips and Kayan advocate the use of bio-absorbable material to form the anchor neither teach any details or methods for effectuating such a device. Criscuolo suggests the use of PG and PL with an absorption time of 2-3 weeks but does not disclose a method of forming the device that results in such an absorption time. In any respect, migration and contraction of the mesh has been documented to occur up to 8 weeks after implant. Loss of fixation after 2 to 3 weeks could well lead to hernia recurrence.

The anchor of the present invention improves the geometry of the anchor described U.S. application Ser. No. 10/709,297 by adding features that minimizes the insertion hole size and while making expulsion from the tissue more difficult. Details of the method of manufacturing the improved anchor are herein provided.

What is needed then is an absorbable mesh fixation anchor and a method of forming an absorbable mesh fixation anchor that exhibits a known absorption time and that exhibits the mechanical properties adequate for the desired fixation strength and the required implant forces.

What is further needed then is an absorbable mesh fixation anchor of improved geometry that collapses to a smaller diameter upon penetrating the mesh and tissue to minimize the entry hole size and an absorbable mesh fixation anchor that flares out to a larger size to resist expulsion when urged proximal.

What is also needed is a method of packaging an absorbable mesh fixation device and the delivery device that minimizes the effects of high ambient shipping temperatures and humidity.

What is also needed is a method of sterilization of an absorbable mesh fixation anchor and its delivery device that has minimal effect on their physical properties, particularly the anchor.

### SUMMARY OF THE INVENTION

A method of producing and deploying a bio-absorbable hernia mesh fixation anchor exhibiting an in vivo absorption time between 1.5 and 13 months and its method of use is disclosed. A method of sterilization and a method of packaging the anchor to retain the critical physical properties of the anchor prior to implantation are also disclosed. The hernia mesh fixation device of the present invention is, preferably, injection molded using any of a variety of mole fractions of d, l-lactide and glycolide co-polymers, depending upon the desired absorption time, and mechanical properties. Preferably the mole ratio is 75-25 percent d, l lactide to glycolide yielding an absorption time after implant of 4-5 months and a glass transition temperature of 49 Deg. C. The modulus of elasticity of the preferred embodiment is 192,000 PSI and the tensile strength is 7200 PSI after injection molding at 150 Deg. C.

The anchor of the present invention is designed with flexibility that allows it to assume a small profile for insertion through the mesh and into the tissue to minimize the mesh entry hole size and to minimize the force required to insert it. Forces that tend to expel the anchor from the tissue such as those generated from sudden increases to IAP causes the tissue snaring elements to expand to a larger diameter thereby increasing the fixation strength of the anchor. It is important to note that the radial forces arising from the resiliency of the tissue acting upon an anchor are large and therefore tend to keep the inserted anchor in a minimal configuration. For this reason it is important that the proximal edges of the tissue snaring elements are angled such that forces that urge the anchor proximal, along the longitudinal axis of the anchor, tend to flare the anchor open much like a toggle bolt. Otherwise, the anchor is held in place only by friction.

The delivery device for the hernia mesh fixation anchor of the present invention is described in detail in U.S. patent application Ser. No. 10/709,297.

Sterilization standards by the U.S. FDA allow radiation doses less than 25 kgy provided the bio-burden is below 1000 colony forming units (CFU). The components of the delivery device and the anchors of present invention are manufactured and assembled under clean room conditions such the bio-burden is well below 1000 CFUs. This allows gamma and E Beam sterilization with doses below the damage threshold of the preferred co-polymers of DLPL and PG, 25 kgy. Mechanical properties of the injected molded anchor of the present invention have been retested after dosing with 25 kgy E Beam. The same values of flex modulus and tensile strength were measured before and after dosing. Gamma or E Beam is the preferred sterilization process, however, an alternate embodiment comprises sterilization employing ethylene oxide without the use of steam and dosed at a temperature below the glass transition temperature.

For the preferred embodiment of the present invention the delivery device loaded with anchors is first sealed into a vacuum formed tray with a breathable Tyvek (a registered trademark of DuPont) lid. This tray is then further hermetically sealed into a foil pouch. The foil pouch is then placed inside an insulated shipping container. The insulation is adequate to assure that the temperature of the anchor remains below 30 deg. C. after exposure to severe heat conditions sometimes experienced during shipping. Gamma or E Beam sterilization is accomplished by radiation through the shipping container.

In an alternate embodiment the sealed vacuum formed tray is placed into the hermetically sealed foil pouch after ETO sterilization. The ETO will penetrate the breathable lid. After the ETO process the device is sealed into the foil pouch and the pouch is placed into the thermally insulated container described above for shipping.

### BREIF DESCRIPTION OF THE DRAWINGS

FIG. 1 is an isometric view of the anchor according to the present invention.

FIG. 2 is a side view of the anchor.

FIG. 3 is a side view of the anchor as it passes through tissue.

FIG. 4 is a side view of the anchor under an expelling force.

### DETAILED DESCRIPTION

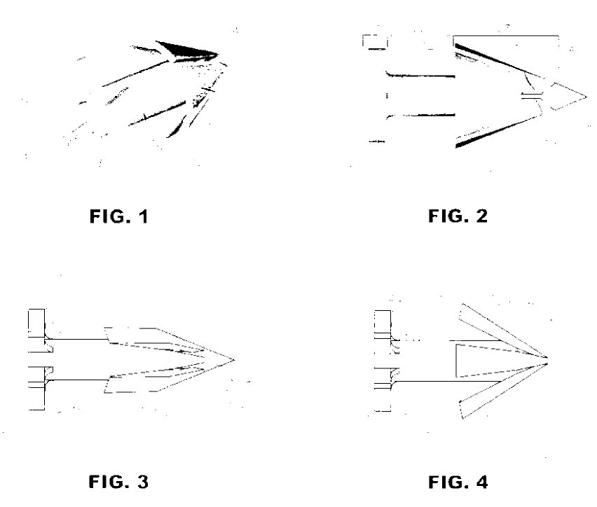

Turning now to FIGS. 1 through FIG. 4, depictions of the anchor of the current invention, generally designated as 10. Anchor 10 comprises three sections, head section 21, mesh-tissue section 22, and tissue snaring section 23. Head section 21 comprises six spokes 11 attached to hub 16. Through-hole 24 is formed parallel with the longitudinal axis of anchor 10. Distal features (not shown) described in U.S. patent application Ser. No. 10/709,297, within through hole 24, serve to restrain anchor 10 distally when anchor 10 comes into contact with tissue penetrator 18 of the delivery device. Head section 21 can alternately be a solid or slotted disk but the spoke arrangement as shown in FIG. 1 aids in injection molding anchor 10 without the need for movable slides in the mold. In either configuration the head section 21 acts to restrain mesh 25 against tissue 26. Mesh-tissue section 12 is generally cylindrical shaped with a dimension transverse to its longitudinal axis that is smaller than the transverse dimension of head 21 and the transverse dimension of tissue snaring section 23. The mesh-tissue section serves to contain the interface of mesh 25 and tissue 26. Owing to the elasticity of tissue 26 upon penetration it is well known that tissue will contract around mesh-tissue section 22 such that tissue 26 will come into contact with the outer wall 12 of mesh-tissue section 22. Tissue snaring section 23 comprises six tissue snares 13 that serve to restrain anchor 10 when anchor 10 is subjected to proximal forces that tend to expel anchor 10 proximally, opposite the direction of tissue penetrator 18. Tissue penetrator 18 is connected to rod 19 that is connected to an actuator in the delivery device as described in detail in co-pending U.S. patent application Ser. No. 10/709,297. Tissue penetrator 18 attached to rod 19, preferably formed from medical grade stainless steel, serves two purposes. Tissue penetrator 18 acts to provide a lead-in for anchor 10 for penetrating mesh 25 and tissue 26 and both tissue penetrator 18 and rod 19 provide added columnar strength to anchor 10. After anchor 10 is set into tissue 26 tissue penetrator 18 and rod 19 are retracted by the actuator mechanism of the delivery device as described in detail in co-pending U.S. patent application Ser. No. 10/709,297. Snares 13 comprise distal ends 14 that smoothly interfaces with tissue penetrator 18 as described in U.S. patent application Ser. No. 10/709,297. Proximal edges 15 of snares 13 are angled with respect to the longitudinal axis of anchor 10 such that under the influence of proximal forces the transverse dimension of tissue snaring section 23 tends to increase. This serves to increase the fixation strength of anchor 10 in tissue 26. The distal end of tissue snaring section 23 is outwardly expandable owing to six slots 17 that allow for retraction of tissue penetrator 18 after anchor 10 is imbedded in tissue 26.

As can be seen in FIG. 3, unlike the anchor described in U.S. patent application Ser. No. 10/709,297, tissue-snaring section 23 is flexible such that snares 13 bend inward toward mesh-tissue outer wall 12 under the radial force of mesh 25 and tissue 26 during penetration. This minimizes the penetration hole diameter in mesh 25 and tissue 26.

FIG. 4 depicts anchor 10 after tissue penetrator 18 has been retracted and with anchor 10 under the influence of a proximal force caused by an increase in intra-abdominal pressure (IAP), for example. In this incidence the transverse dimension of tissue snaring section 23 increases, as shown in FIG. 4, such that the fixation strength of anchor 10 increases.

Five embodiments of anchor 10 are described herein comprising four different molar ratios of DLPL and PG. The resins of the co-polymers in each case were prepared using well-known techniques of polymerization of cyclic dimmers. The molar percentages (M) of DLPL and PG were measured along with the residual monomer percentage (RM). After polymerization the resins were thoroughly dried. Anchor 10 was then injection molded in a standard micro-molding machine at 150 Deg. C. The transition glass temperature (Tg), the absorption time at 37 Deg. C. (to 20% of the original mass) (AT), the tensile strength (TS) and Young's modulus (YM) were then measured. Anchor 10 was then subjected to 25 kgy E Beam radiation and the tensile strength and Young's modulus re-measured. Standard techniques, well known by those skilled in the art, were employed in the measurements of each of the parameters. The results are shown below:

Case I

| | M, | M, | | Tg, | | | |
|---|---|---|---|---|---|---|---|
| | DLPL, | PG, | RM, | Deg. | AT, | TS, | YN, |
| Parameter | % | % | % | C. | Months | PSI | PSI |
| | 100 | 0 | 2.1 | 49.4 | 13 | 6100 | 206,000 |

Case II

| | M, | M, | | Tg, | | | |
|---|---|---|---|---|---|---|---|
| | DLPL, | PG, | RM, | Deg. | AT, | TS, | YN, |
| Parameter | % | % | % | C. | Months | PSI | PSI |
| | 85 | 15 | 2.1 | 49.7 | 5.8 | 7900 | 198,000 |

Case III

| | M, | M, | | Tg, | | | |
|---|---|---|---|---|---|---|---|
| | DLPL, | PG, | RM, | Deg. | AT, | TS, | YN, |
| Parameter | % | % | % | C. | Months | PSI | PSI |
| | 75 | 25 | 1.6 | 49.1 | 4.3 | 7200 | 192,000 |

Case IV

| | M, | M, | | Tg, | | | |
|---|---|---|---|---|---|---|---|
| | DLPL, | PG, | RM, | Deg. | AT, | TS, | YN, |
| Parameter | % | % | % | C. | Months | PSI | PSI |
| | 65 | 35 | 1.9 | 47.2 | 3.2 | 74000 | 190,000 |

Case V

| | M, | M, | | Tg, | | | |
|---|---|---|---|---|---|---|---|
| | DLPL, | PG, | RM, | Deg. | AT, | TS, | YN, |
| Parameter | % | % | % | C. | Months | PSI | PSI |
| | 52 | 48 | 1.2 | 46.7 | 1.5 | 8100 | 188,000 |

In each case retesting the tensile strength and Young's modulus after subjecting the anchor 10 to 25 kgy E Beam radiation yielded results statistically indistinguishable from the values in the tables above.

To design an appropriate insulated shipping container the historical average daily temperatures over a “hot weather route” from Florida to Arizona were obtained from www.engr.udayton.edu/weather. Heat flux data were determined from the historical data resulting in an insulation requirement of 2.5 inches of Cellofoam (a registered trademark of Cellofoam of North America, Inc.) with a thermal R value of 3.86 per inch of thickness. Anchors 10 were then shipped over the route packed in the insulated container and the internal temperature of a un-air conditioned cargo space of a roadway common carrier was measured during a five-day trip from Jacksonville Fla. to Phoenix Ariz. from September 9 till Sep. 14, 2004. The internal temperatures of the cargo space, Tc, and the internal temperature of the insulated container, Ti, containing anchors 10 were recorded every 30 minutes. The minimum and maximum temperatures in the cargo space and the insulated container are shown below:

| | Day 1 | Day 2 | Day 3 | Day 4 | Day 5 |
|---|---|---|---|---|---|
| Maximum Tc | 37 | 34 | 29 | 48 | 50 |
| Minimum Tc | 24 | 18 | 15 | 27 | 27 |
| Maximum Ti | 27 | 27 | 26 | 27 | 27 |
| Temperature, | | | | | |
| Minimum Ti | 24 | 26 | 21 | 24 | 24 |
| Temperature, | | | | | |

The above temperatures are degrees centigrade.

Thus it is seen from the data above that the insulated shipping container is adequate for maintaining anchor 10 temperatures well below the glass transition temperature of 49 Deg. C. of the preferred co-polymer, 75/25 DLPL/PG, Case III above.

The preferred embodiment for the current invention is an injection molded anchor as depicted in FIG. 1 comprising 75% DLPL, 25% PG, sterilized with radiation, either gamma or E Beam, at 25 kgy and packaged first in a hermetically sealed pack and an insulated shipping container.

From the foregoing, it will be appreciated that the absorbable anchor of the present invention functions to securely fasten mesh to tissue and an anchor that will disintegrate after the body has secured the mesh against migration and contraction. The absorbable anchor of the present invention can be sterilized so that mechanical properties are maintained and it can be shipped under severe temperature conditions with insulated packaging so that the glass transition temperature is not exceeded. It will also be appreciated that the absorbable anchor of the present invention may be utilized in a number of applications such as hernia repair, bladder neck suspension, and implant drug delivery systems.

While several particular forms of the invention have been illustrated and described, it will be apparent by those skilled in the art that other modifications are within the scope and spirit of the present disclosure.

What is claimed is:

1. A method of producing and deploying a surgical anchor for anchoring mesh to tissue comprising:
forming the anchor from at least one bio-absorbable polymer,
providing a surgical anchor delivery device equipped with a tissue penetration element,
loading the anchor into the delivery device,
sterilizing the anchor at a temperature below the glass transition temperature of the polymer,
packaging the anchor and the delivery device in a hermetically sealed package,
delivering the anchor and delivery device to a surgical site further packaged in an insulated container such that the anchor temperature does not exceed the glass transition temperature of the polymer,
removing the delivery device and the anchor from the insulated container and the hermetically sealed package,
inserting the delivery device and the anchor into a surgical field,
penetrating tissue with the tissue penetration element and the anchor, and
imbedding the anchor into the tissue.

2. The method according to claim 1 wherein the bio-absorbable polymer is a homo polymer of either polylactide or polyglycolide or co-polymer of polylactide and polyglycolide.

3. The method according to claim 1 wherein the bio-absorbable polymer is a co-polymer of polylactide and polyglycolide with a molar content of polylactide ranging, preferably, from 50 to 100 percent.

4. The method of claim 1 wherein the anchor polymer exhibits a Young's modulus in the range of 150,000 to 2,000,000 PSI.

5. The method of claim 1 wherein the anchor exhibits a tensile strength in the range of 5,000 to 10,000 PSI.

6. The method of claim 1 wherein the anchor polymer exhibits an absorption time in vivo between 1.5 and 14 months.

7. The method of claim 1 wherein the anchor exhibits a glass transition temperature in the range of 40 to 60 degrees centigrade.

8. The method according to claim 1 wherein sterilization is effectuated using ethylene oxide.

9. The method according to claim 1 wherein sterilization is effectuated using gamma radiation.

10. The method according to claim 1 wherein sterilization is effectuated using electron beam radiation.

11. The method according to claims 5 and 6 wherein the radiation level is, preferably, equal to 25 kgy or less.

12. A mesh anchor for penetrating tissue and fixating the mesh having a longitudinal axis comprising:
A head section,
A mesh-tissue retaining section, and
Tissue penetrating and snaring elements having a dimension transverse to the longitudinal axis that is urged smaller during the tissue penetration and urged larger during tissue snaring.

13. The mesh anchor according to claim 1 wherein the proximal edges of the tissue penetrating and snaring elements are angled in such a manner as to cause the transverse dimension of the tissue penetrating and snaring elements to increase when the anchor is urged proximal.

14. The mesh anchor according to claim 1 wherein the anchor comprises a bio-absorbable polymer, either a homo polymer of either polylactide or polyglycolide or co-polymer of polylactide and polyglycolide.

15. The mesh anchor according to claim 1 wherein the anchor polymer exhibits a young's modulus in the range of 150,000 to 2,000,000 PSI.

16. The mesh anchor according to claim 1 wherein the anchor exhibits a tensile strength in the range of 5,000 to 10,000 PSI.

17. The mesh anchor according to claim 1 wherein the anchor polymer exhibits an absorption time in vivo between 1.5 and 14 months.

18. The mesh anchor according to claim 1 wherein the anchor exhibits a glass transition temperature in the range of 40 to 60 degrees centigrade.
